(19) United States
(12) Patent Application Publication
(10) Pub. No.: US 2006/0100510 A1
(43) Pub. Date: May 11, 2006

(54) Method and apparatus for using tomography for placement of an instrument

(76) Inventor: Remy Klausz, Neuilly sur Seine (FR)

Correspondence Address: CANTOR COLBURN, LLP, 55 GRIFFIN ROAD SOUTH, BLOOMFIELD, CT, 06002, US

(21) Appl. No.: 11/181,455

(22) Filed: Jul. 14, 2005

(30) Foreign Application Priority Data
Oct. 21, 2004 (FR) 04 11205

(51) Int. Cl. A61B 5/05

(52) U.S. Cl. 600/429

(57) ABSTRACT

A method and apparatus for using a tomography to facilitate the placement of an instrument in a vessel or in an organ of a patient's body wherein a specific or selected region of the patient is defined. X-ray radiation is projected onto the selected region, the X-ray radiation being emitted by the X-ray source of the apparatus. The radiation transmitted to a detector of the apparatus is measured, the detector being positioned in line with the X-ray source. The signals of the selected region are measured by the detector and transmitted for acquisition that stores the image. The acquired signals for the image are transmitted to a display in order to display a projected image of the selected region of the patient in a plane parallel to the longitudinal axis of the patient, such that the time interval between measuring the radiation and displaying the radioscopic image is short enough to allow the instrument to be guided in the patient's body in “real-time”. The method is repeated in order to acquire new projected images of the selected region.

### CROSS-REFERENCE TO RELATED APPLICATIONS

This application claims the benefit of a priority under 35 USC 119 (a)-(d) to French Patent Application No. 04 11 205 filed Oct. 21, 2004, the entire contents of which are hereby incorporated by reference.

### BACKGROUND OF THE INVENTION

An embodiment of the present invention relates to a method of using a tomography device generally referred to as an X-ray tomodensitometer or the like, for obtaining radioscopic images to facilitate the placement of an instrument in a vessel or in an organ of an object, such as a patient's body, while maintaining a normal operating mode. An embodiment of the invention also relates to a tomography apparatus for carrying out the method.

A computerized tomographic apparatus, commonly referred to as tomodensitometers or CT scanners, make it possible to reconstruct images corresponding to the value of the linear attenuation coefficient at any point of a cut or cross section, are well known. These apparatus conventionally comprise a means for providing a source of radiation, such as an X-ray source, and means for detecting the radiation, such as an array of detectors for the emitted X-rays, which defines the field of the tomodensitometer The X-ray source and the detectors can be integrated with a mobile support which can move in rotation about an annular stand, the axis of rotation of the mobile support generally coinciding with the longitudinal axis of the object or patient. A horizontal support, referred to as a table, on which the object or patient is positioned can be moved along the longitudinal axis of the object or patient in order to cross the field of the tomodensitometer. These apparatus furthermore comprise means for control that control the X-ray source, means for acquisition means that receive the information transmitted by the detectors and means for reconstruction for forming an image, which is transmitted to a means for display or image storage.

A known “fluoroscanner”, such as a tomodensitometer marketed by the assignee, General Electric Company, as the Smartview®, positions an instrument in the body of the object or a patient's body for a diagnostic or interventional procedure. This type of system allows cross-sectional images to be formed at a high rate, of the order of 6 to 12 images per second, making it possible to guide the positioning of an instrument in a patient's body. This type of system, however, has the drawback of exposing the patient to large radiation doses.

Furthermore, it is known to use a tomodensitometer to carry out diagnostics by cinedensigraphy, for example, as described in FR 2 461 485. This patent describes a method of using an X-ray tomodensitometer that, according to the technique of cinedensigraphy, obtains information characteristic of a variation as a function of time in the shape and/or density of parts of a subject that is irradiated with X-rays. The method comprises subjecting each section for examination to X-rays and measuring the radiation transmitted to a detector that provides signals corresponding to the absorption respectively experienced by the elementary beams when they cross the section at the time of this first measurement. This measurement is repeated several times in succession so as to obtain the values taken by the signals for the same projection, that is to say, at a constant X-ray incidence with respect to the section to be examined, at different times, thus sampling this projection over time. The successions of signals obtained in this way are processed and stored. The amplitude of the successions of the signals that are obtained, as a function of time for one or more given elementary beams, and/or at a constant time for the various elementary signals, can then be displayed.

In order to perform an angiography, it is generally necessary to inject a contrast agent opaque to X-rays, and then take images. These images may comprise radiographic images taken by using an angiography device or tomographic images in reconstructed cross section, taken by means of a tomodensitometer. The contrast agent may be injected either intravenously for nonselective display of the organs and/or vascular subdivisions, or intra-arterially for selective display. Whether the images are taken by means of an angiography device or a tomodensitometer, the technique of intravenous injection presents the risk of causing damage to the patient's veins at the point of injection. This is because the contrast agent is injected rapidly, that is to say with a high pressure, in order to maintain an elevated concentration for as long as possible. This technique of intravenous injection furthermore presents the drawback, besides propagation of the contrast agent opaque to X-rays throughout the patient's organs, of requiring a large quantity of the contrast agent.

For images taken by means of a tomodensitometer, the technique of intravenous injection generates artefacts on the images. For a cardiac angiography, in particular, the concentration of the contrast product present in the venous segment passing close to the heart is higher than the concentration of the contrast product actually present in the heart, and this creates artefacts which degrade the image of the heart. The transit time of the contrast product, being at least equal to the acquisition time of the images, may furthermore lead to confusion between the arterial phase and the parenchymatic phase when interpreting the image. Further, in view of the good contrast resolution offered by tomodensitometers, an intra-arterial and therefore selective injection is generally considered to be unnecessary, so that cardiac angiographies are carried out intravenously.

In order to overcome these drawbacks, in view of the low contrast resolution of angiographic devices, it is well known in projection angiography to inject the contrast product directly into the organ to be examined, or into a vessel leading into the organ, such as the aorta which leads into the heart and supplies the coronary arteries. Such an injection, however, requires the assistance of a radioscopy device in order to position the instrument in the organ or in the vessel prior to the injection.

If the same technique is applied to tomodensitometry procedures, it is possible to use a mobile radioscopy device, for example, such as the radioscopy device marketed under the brand “OEC 9800 Cardiac” by the assignee, General Electric Company, or the device as described in EP 0 231 969. One alternative comprises using a mobile table to move the patient between a fixed radioscopy device and a tomodensitometer, and vice versa, such as the equipment marketed under the brand “ATOM” by the assignee, General Electric Company. These types of devices present the drawback of being expensive and bulky, and are therefore are not in very widespread use.

### BRIEF DESCRIPTION OF THE INVENTION

An embodiment of the invention seeks to overcome these drawbacks by providing a method and apparatus for using a tomodensitometer to obtain radioscopic images to facilitate the placement of an instrument in an object, such as a vessel or in an organ of a patient's body, while maintaining a normal operating mode.

An embodiment of the invention relates to a method comprising: defining a specific region of the object, referred to as the selected region; projecting radiation onto the selected region, the radiation being emitted by means for providing a source of radiation from the tomodensitometer; measuring the radiation transmitted to means for detection of the tomodensitometer, which are positioned in line with the radiation source; transmitting signals measured by the means for detection in the selected region to means for acquisition, which store the image and which transmit the image to means for display in order to display a projected image of the selected region of the object in a plane parallel to the longitudinal axis of the object, such that the time interval between measuring the radiation and displaying the radioscopic image is short enough to allow the instrument to be guided in the object in “real-time”, that is to say a time interval generally shorter than 1 second; and repeating the preceding steps in order to acquire new projected images of the selected region.

To facilitate determination of the so-called selected region, the method comprises, prior to the acquisition of the projected radioscopic images of the selected region, forming a radiograph of the object or patient by projecting radiation emitted by the radiation source and by simultaneously moving an object or patient table along the longitudinal direction of the table, the signals measured by the means for detection being transmitted to means for acquisition of the signals, which store them and which can later transmit them to means for display in order to display the silhouetted radiograph of the object or patient. A silhouette radiographic image of the object or patient can be obtained according to the ScoutView® mode of the tomodensitometers marketed by the assignee, General Electric Company. For the sake of simplicity and clarity, a silhouette radiographic image of the object or patient will be referred to as a ScoutView® radiographic image in this application. The projected image of the selected region of the patient is preferably overlaid on the ScoutView(® radiographic image.

Furthermore, the acquisition of new projected radioscopic images of the selected region may be carried out either by the operator, who actuates a control means, or repetitively at regular intervals.

In addition, the selected region is moved along the longitudinal axis of the ScoutView® radiographic image, between two successive radioscopic images of the selected region, in order to monitor the positioning of the instrument. The selected region is moved either by modifying the position of the reference or references, which delimit the width of the selected region, along the longitudinal axis of the ScoutView® radiographic image, or by moving the table continuously along its longitudinal axis or with a predetermined increment, which is recorded in a means for control, along the longitudinal axis of the ScoutView® radiographic image. An embodiment of the method permits acquiring radioscopic images in projection to assist the operator when placing a catheter, for example, without requiring the use of a specific radioscopy device.

An embodiment of the invention also relates to a tomography apparatus, generally referred to as a tomodensitometer, having means for horizontal support referred to as a table, on which an object or patient is positioned, means for providing a source of radiation, such as an X-ray source, capable of emitting X-rays and means for mobile rotation mounted on a support which can move in rotation about an axis of rotation generally coinciding with the longitudinal axis of the patient who is lying on the table. The apparatus comprises a means for detection of the radiation that can be integrated with the mobile rotation support, facing the radiation source, means for control, means for acquiring the signals transmitted by the means for detection, means for image reconstruction and means for display. The means for acquisition can process either a succession of data relating to a region of the object or patient which are acquired by the means for detection during rotation of the mobile support of the radiation source so that the means for reconstruction generates a so-called tomographic image in a so-called tomographic mode, or a succession of data acquired by moving the table along the longitudinal direction of the table without rotating the mobile support of the X-ray source in order to form a ScoutView® radiographic image in a so-called radiographic mode. The method comprises means for selection between the tomographic mode or the radiographic mode and a radioscopic mode in which the images displayed are successive radioscopic images, projected in a constant direction, of the selected region of the object or patient in a plane parallel to the longitudinal axis of the object or patient.

### BRIEF DESCRIPTION OF THE FIGURES

Other advantages and characteristics will be understood more clearly from the following description of several alternative embodiments, given by way of nonlimiting examples, of the method of using a tomodensitometer and of a tomodensitometer for carrying out the method, based on the appended drawings in which:

FIG. 1 is a schematic perspective view of a tomodensitometer for carrying out the method according to an embodiment of the invention;

FIG. 2 is a representation of a radioscopic image of the selected region, overlaid in a ScoutView® radiographic image; and

FIG. 3 is a representation of a second radioscopic image of the selected region, overlaid on the ScoutView® radiographic image, after the selected region has been moved.

### DETAILED DESCRIPTION OF THE INVENTION

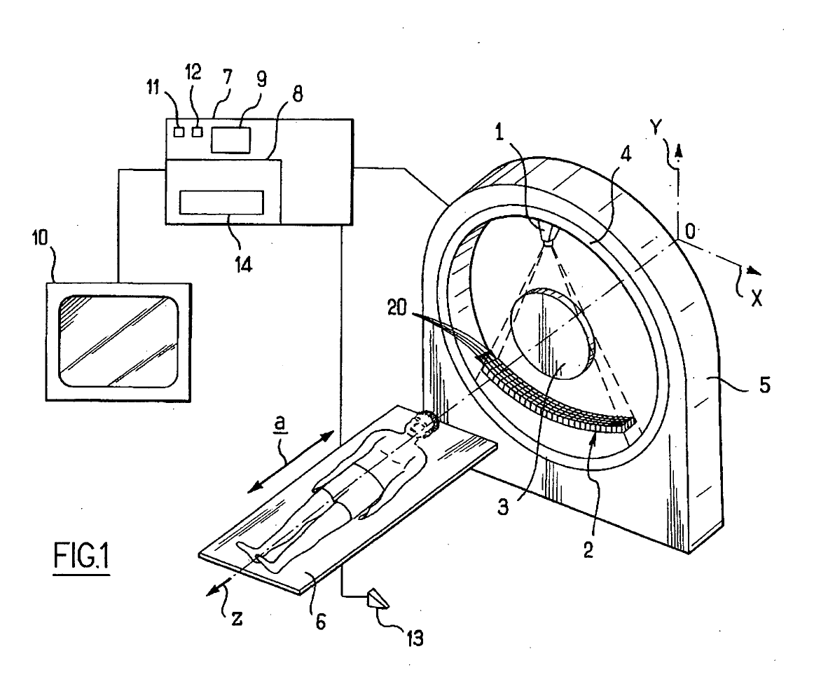

Referring to FIG. 1, the tomodensitometer has an X-ray source 1, usually comprising an X-ray tube, and means 2 for detecting the emitted X-rays which define the field 3 referred to as FOV (Field Of View) of the tomodensitometer. The X-ray source I and the means for detection 2 can be integrated with a means for support 4 which can move in rotation about a vertical annular stand 5 defining a plane (Ox, Oy), the axis of rotation of the mobile support 4 generally coinciding with the longitudinal axis of the patient (Oz). The X-ray source I produces an X-ray beam, which is spread in the form of a fan and which strikes the curved means for detection 2, the concavity of which faces the focus of the X-ray source 1.

The means for detection 2 may comprise flat detection means, without departing from the scope and extent of the disclosed embodiments of the invention.

In order to provide radioprotection for the operator, the X-ray source I comprises suitable collimation means (not shown in FIG. 1) for restricting the width of the field 3 in the direction perpendicular to the longitudinal axis of the patient when taking images in projection, that is to say radiographic images or radioscopic images as will be described below.

The tomodensitometer furthermore comprises a horizontal support referred to as a table 6, which can be moved in a to-and-fro movement by motorized means (not shown in FIG. 1) along an axis z orthogonal to the plane (Ox, Oy) of the stand 5 as indicated by the double arrow a, so that the patient crosses the field 3 of the tomodensitometer during to-and-fro movements of the table 6. It will be noted that the table 6 may also be moved vertically, along the axis (Oy), by suitable means (not shown in FIG. 1). The tomodensitometer furthermore comprises means for control 7 that control the X-ray source 1 in particular, means for slaving the table 6 and the mobile support 4, and means for acquisition 8 which receive the signals measured by the means for detection 2. The tomodensitometer also comprises means for reconstruction 9 which comprises a computer program including an algorithm for processing the signals, for example, and which generate a transverse tomographic image.

The tomodensitometer comprises a first operating mode referred to as tomographic, in which the means for control 7 control the X-ray source 1, the rotation of the mobile support 4 about the stand 5 and the emission of the X-rays, which are received by the means for detection 2, the patient's region to be examined being already placed in the field 3 of the tomodensitometer by moving the table along the axes Oz and/or Oy. The output signals of the means for detection 2 are then transmitted to the means for acquisition 8, which transmit to the means for reconstruction 9 that, in a known fashion, generate a transverse axial tomographic image that is transmitted to the means for display 10.

The tomodensitometer comprises a first means 11 for selecting a so-called radiographic mode, in which the means for control 7 control the X-ray source 1 so that it emits X-rays without rotation of the mobile support 4 and move the patient table 6 along the z axis. The means for detection 2 transmit the signals corresponding to the X-rays to the means for acquisition 8. For specific positions of the table 6, therefore, measurement values are obtained which characterize the attenuation of the X-radiation as it passes through the patient. On the basis of these measurements, the means for acquisition 8 determine a silhouetted radiographic image, referred to as a ScoutView® radiographic image, which is transmitted to the means for display 10.

The tomodensitometer comprises a second means 12 for selecting a so-called radioscopic mode, in which the images displayed are successive so-called radioscopic images in projection of a selected region of the patient in a plane parallel to the longitudinal axis of the said patient, as will be described in detail below. The tomodensitometer furthermore comprises a means for controlling the acquisition of the radioscopic images, the means for control preferably comprising a pedal 13 positioned close to the table 6. Optionally, the means for control 7 of the tomodensitometer comprise means for determining the radioscopic parameters, namely the high voltage applied to the X-ray tube as well as the strength of the current flowing through the tube, and the length of the pulses in the case of pulsed X-ray emission, as a function of the characteristics of the ScoutView® radiographic image acquired previously. Alternatively, the means for control 7 comprise means for determining the radioscopic parameters for the acquisition of a new radioscopic image as a function of the characteristics of the radioscopic image acquired previously by the tomodensitometer, or as a function of the characteristics of the successive radioscopic images acquired previously.

It is clear that the tomodensitometer may comprise other means for control, such as other pedals positioned close to the table 6, in order, for example, to make it possible to acquire a ScoutView® radiographic image by actuating one pedal and to move the table 6 along the z axis by actuating another pedal.

In this particular exemplary embodiment, the means for detection 2 of the tomodensitometer comprise a plurality of rows 20 of X-ray detectors extending along the longitudinal axis z of the table 6, so that the field 3 has a large width of the order of 40 mm, for example, which is the same as the field of the tomodensitometer marketed under the brand LightSpeed VCT by the assignee, General Electric Company. It is clear, however, that the means for detection 2 of the tomodensitometer may comprise a single row of detectors accompanied by collimation of the X-rays so as to generate images having an acceptable resolution.

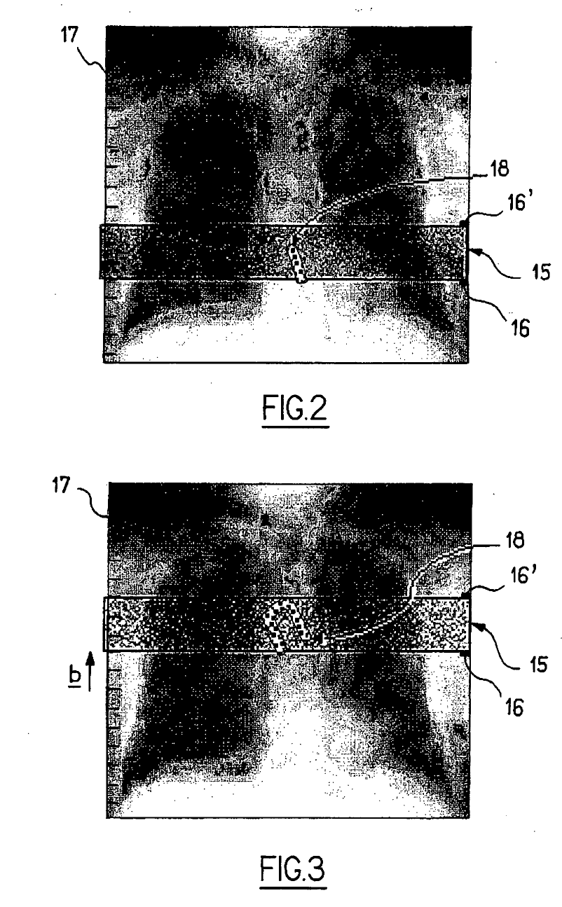

After having actuated the means 12 for selecting the radioscopic mode, referring to FIG. 2, the method of using a tomodensitometer comprises defining a specific region of the patient, referred to as the selected region 15, so as to project X-rays onto the selected region 15. The radiation transmitted to the detection means 2 of the tomodensitometer which are positioned in line with the X-ray source 1 is measured. The signals measured by the detection means 2 in the selected region 15 are transmitted to the acquisition means 8, which store the image and which transmit the image to the means for display 10 in order to display a projected radioscopic image of the selected region 15 of the patient in a plane parallel to the longitudinal axis of the patient. The projected radioscopic image of the selected region 15 of the patient is overlaid in the ScoutView® radiographic image. In order to provide “real-time” guidance for placing the instrument in the patient's body, the tomodensitometer may perform the steps as described above for each new image, the time interval between measuring the radiation and displaying the radioscopic image being short enough, that is to say generally less than I second, and preferably between 10 and 15 hundredths of a second.

To facilitate determination of the selected region 15, the method comprises, a step of forming a ScoutView® radiographic image of the patient by projecting X-rays from the X-ray source 1, which is locked in rotation, and by simultaneously moving the patient table 6 along the longitudinal direction z of the table 6, the signals measured by the detection means 2 being transmitted to the means 8 for acquisition of the signals, which store the ScoutView® radiographic image in a memory unit 14 and which transmit the radiographic image to the means for display 10.

In an alternative embodiment, referring to FIG. 2, the selected region 15 may be defined by positioning a reference 16 along the longitudinal axis of the silhouetted radiographic image displayed on the means for display 10, the width of the selected region 15 being predetermined, and preferably by positioning two references 16, 16′ along the longitudinal axis of the silhouetted radiographic image displayed on the display means 10, the width of the selected region 15 being equal to the distance between the two references 16, 16′.

In another alternative embodiment, the selected region 15 is defined by a graphical scale that is shown on the means for display 10, the reference point of which is set to a user-determined position of the table 6 along the z-axis.

It is clear that the selected region may also be defined by using a radiopaque scale 17 which is positioned under the table 6, the image of which is formed on the display means, one of the graduations of the scale defining a reference point.

In the radioscopic mode, and for a tomodensitometer whose means for detection 2 comprise a single row 20 of detectors or a limited number of rows 20 of detectors, the patient table 6 is moved longitudinally, that is to say along the z axis, over a distance generally equal to the width of the selected region 15 when acquiring the projected radioscopic images of the selected region 15, obtained firstly by projecting the X-rays onto the selected region 15 then secondly by measuring the radiation transmitted to the means for detection 2 positioned in line with the X-ray source 1.

The projected radioscopic images of the selected region 15 of the patient can be acquired at a regular interval. The refresh time, that is to say the time between the acquisition of two successive radioscopic images, then depends on the width of the selected region 15 and the movement rate of the table 6. For example, for a tomodensitometer whose means for detection 2 comprise a single row of detectors 20, the refresh time is about 0.6 s for a width of the selected region 15 equal to 40 mm and for a movement rate of the-table 6 equal to 70 mm/s. In the same way, for a tomodensitometer whose means for detection 2 comprise a plurality of rows of detectors 20 having a width of 20 mm, the refresh time is about 0.3 s for a width of the selected region 15 equal to 40 mm and for a movement rate of the table 6 equal to 70 mm/s. It will be observed that the refresh time is much shorter for a tomodensitometer comprising a plurality of rows of detectors 20 and that, for a constant width of the selected region 15, the refresh time is in general commensurately shorter as the width of the rows of detectors 20 is large.

It is clear that in order to increase the frequency of the image acquisition, the table 6 may be moved alternately in both directions of the z axis, as indicated by the arrow a in FIG. 1, within the limits of the width of the selected region 15.

In another alternative embodiment, the acquisition of the projected images of the selected region 15 are controlled by the operator, who actuates a means for control such as a pedal 13, for example. By actuating the pedal 13, the operator initiates the acquisition of a succession of radioscopic images for a specific time interval, such as an interval of a few seconds. At the end of this time interval, the operator may actuate the pedal 13 again in order to initiate the acquisition of a new succession of radioscopic images. If the operator does not need any more radioscopic images before the end of the time interval, he or she may stop the acquisition of the radioscopic images by releasing the pedal 13 in order to limit the irradiation of the patient.

For a tomodensitometer whose means for detection 2 comprise a plurality of rows of detectors so as to provide a wide field 3, such as the LightSpeed VCT tomodensitometer in which the width of the rows of detectors is 40 mm, the patient table 6 will not need to be moved longitudinally during acquisition of the projected images of the selected region 15 if the latter has a width less than or equal to 40 mm. It will be noted that in contrast to tomodensitometers which have a single row of detectors 20 or a limited number of rows of detectors 20, for which a to-and-fro movement of the table 6 liable to distract the user when placing the instrument in the patient's body is necessary for acquisition of the radioscopic images, these wide-field tomodensitometers make it possible to acquire the radioscopic images without moving the table 6 if the width of the radioscopic image is less than or equal to the width of the rows of detectors 20. Such tomodensitometers furthermore permit an image refresh time of the order of one millisecond. If a longer refresh time is desired, it is sufficient either to reduce the acquisition rate of the signals at the detectors of the means for detection 2 or to add up the successive elementary projected radioscopic images over a fixed number or using a recursive algorithm, or to emit pulsed X-rays from the X-ray source in the manner of pulsed radioscopy techniques.

Furthermore, referring to FIGS. 2 and 3, the selected region 15 may be moved along the longitudinal axis of the silhouetted radiographic image of the patient between two successive radioscopic images of the selected region 15, in order to monitor the positioning of a catheter 18, as indicated by the arrow b in FIG. 3. The selected region 15 may be moved along the longitudinal axis of the ScoutView® radiographic image either by modifying the position of the reference or references 16, 16′ or by moving the table 6 continuously along its longitudinal axis (Oz) or with a predetermined increment, which is recorded in the control means 7. The increment may be equal to a fraction of the width of the selected region 15, or it may be more than the width of the selected region 15.

In order to improve the quality of the image, the radioscopic images may be generated according to the “road-mapping” system well known to the person skilled in the art, which comprises subtracting each current radioscopic image with an image of the selected region 15 previously taken and recorded in the memory unit 14 of the means for acquisition 8 with a contrast product highlighting the patient's vascular network.

One or more of the radioscopic parameters, namely the high voltage applied to the tube of the X-ray emission means I as well as the strength of the current flowing through the tube and the length of the radiation, are determined so that, on the one hand, the radioscopic images have a satisfactory contrast and an acceptable noise level and, on the other hand and quite independently, the patient is exposed to a radiation dose less than or equal to a specific threshold. This threshold may be determined by the standards or regulations in certain countries, for example a threshold of between 50 and 200 mGy/min. The radioscopic parameters are, for example, determined by the means for control 7 from the characteristics of the ScoutView® radiographic image.

Alternatively, one or more of the radioscopic parameters may be determined by the means for control 7 from the characteristics of one or more successive images of the selected region 15. For instance, the radioscopic parameters for the acquisition of a new projected radioscopic image are determined from the characteristics of the projected radioscopic image acquired previously, or alternatively from the characteristics of the previous projected radioscopic images.

Some or all of the succession of the radioscopic images may be recorded in the memory unit 14 in order to be displayed after the instrument has been placed in the patient's body, for example for checking purposes.

The method of the disclosed embodiments or equivalents may be suitable for the placement of an instrument in any of a patient's organs, in a cyst, in a tumour, in a vessel, which may be a vein or an artery, for example the aorta, or alternatively in the biliary channels in order to prepare for a retrograde cholangiography by endoscopy, in particular, and that the examples given above are no more than particular illustrations implying no limitation with respect to the fields of application of the embodiments of the invention.

In addition, while an embodiment of the invention has been described with reference to exemplary embodiments, it will be understood by those skilled in the art that various changes may be made in the way and/or structure and/or function and/or result and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. Moreover, the use of the terms first, second, etc. do not denote any order or importance, but rather the terms first, second, etc. are used to distinguish one element from another. In addition, the order of the disclosed steps is exemplary. Furthermore, the use of the terms a, an, etc. do not denote a limitation of quantity, but rather denote the presence of at least one of the referenced item.

What is claimed is:

1. A method of using a tomography apparatus to facilitate the placement of an instrument in a body of an object comprising::
defining a specific or selected region of the object;
projecting radiation onto the selected region from means for providing a source of radiation;
measuring the radiation transmitted to a means for detection;
transmitting the signals measured by the means for detection in the selected region to means for acquisition, which store the image;
transmitting the acquired signals to means for display in order to display a projected image of the selected region of the object in a plane parallel to the longitudinal axis of the object, such that the time interval between measuring the radiation and displaying the radioscopic image is short enough to allow the instrument to be guided in the body in “real-time” and
repeating the preceding steps in order to acquire new projected images of the selected region.

2. The method according to claim 1 wherein prior to the acquisition of the projected images of the selected region comprising:
forming a radiograph of the object by projecting radiation and simultaneously moving means for support of the object along a longitudinal direction of the means for support;
transmitting the signals measured by the means for detection to the means for acquisition of the signals, which store a silhouette radiograph of the object; and
transmitting the radiograph to the means for display.

3. The method according to claim 2 wherein the projected radioscopic image of the selected region of the object is overlaid on the image of the silhouette radiograph of the object.

4. The method according to claim 1 wherein the acquisition of the projected radioscopic image of the selected region is controlled by an operator who can actuate a means for control.

5. The method according to claim 2 wherein the acquisition of the projected radioscopic image of the selected region is controlled by an operator who can actuate a means for control.

6. The method according to claim 3 wherein the acquisition of the projected radioscopic image of the selected region is controlled by an operator who can actuate a means for control.

7. The method according to claim 1 wherein the projected radioscopic images of the selected region of the object are acquired and transmitted to the means for display means at regular intervals.

8. The method according to claim 2 wherein the projected radioscopic images of the selected region of the object are acquired and transmitted to the means for display means at regular intervals.

9. The method according to claim 3 wherein the projected radioscopic images of the selected region of the object are acquired and transmitted to the means for display means at regular intervals.

10. The method according to claim 7 wherein the selected region is defined by positioning at least one reference along a longitudinal axis of the silhouette radiographic image displayed on the means for display.

11. The method according to claim 8 wherein the selected region is defined by positioning at least one reference along a longitudinal axis of the silhouette radiographic image displayed on the means for display.

12. The method according to claim 9 wherein the selected region is defined by positioning at least one reference along a longitudinal axis of the silhouette radiographic image displayed on the means for display.

13. The method according to claim 10 wherein a width of the selected region is predetermined.

14. The method according to claim 11 wherein a width of the selected region is predetermined.

15. The method according to claim 12 wherein a width of the selected region is predetermined.

16. The method according to claim 7 wherein the selected region is defined by positioning two references along a longitudinal axis of the silhouetted radiographic image displayed on the means for display, the width of the selected region being equal to the distance between the two references.

17. The method according to claim 8 wherein the selected region is defined by positioning two references along a longitudinal axis of the silhouetted radiographic image displayed on the means for display, the width of the selected region being equal to the distance between the two references.

18. The method according to claim 9 wherein the selected region is defined by positioning two references along a longitudinal axis of the silhouetted radiographic image displayed on the means for display, the width of the selected region being equal to the distance between the two references.

19. The method according to claim 1 wherein the selected region is moved along a longitudinal axis of the silhouette radiographic image of the object, between two successive radioscopic images of the selected region, in order to monitor the positioning of the instrument.

20. The method according to claim 19 wherein the selected region is moved by modifying the position of the reference or references along the longitudinal axis of a silhouette radiographic image of the object.

21. The method according to claim 19 wherein the selected region is moved with a predetermined increment, which is recorded in the means for acquisition, along the longitudinal axis of the silhouette radiographic image of the object.

22. The method according to claim 21 wherein the increment is equal to a fraction of the width of the selected region.

23. The method according to claim 21 wherein the increment is more than the width of the selected region.

24. The method according to claim 19 wherein the selected region is moved by moving the means for support continuously along its longitudinal axis.

25. The method according to claim 1 wherein means for support is moved longitudinally along a z axis, over a distance generally equal to the width of the selected region.

26. The method according to claim 1 wherein one or more radioscopic parameters is determined so that the object is exposed to a radiation dose less than or equal to a specific threshold.

27. The method according to claim 26 wherein one or more of the radioscopic parameters of the irradiation are determined by the means for control from the characteristics of the silhouette radiographic image of the object.

28. The method according to claim 26 wherein one or more of the radioscopic parameters are determined by the means for control from the characteristics of successive radioscopic images of the selected region.

29. The method according to claim 1 comprising:
subtracting each current radioscopic image with an image of the selected region previously taken and recorded in the means for acquisition with a contrast product highlighting a vascular network of the object.

30. A tomographic apparatus comprising:
means for horizontal support on which an object is positioned;
means for providing a source of radiation;
means for providing a support that can move in rotation about an axis of rotation generally coinciding with a longitudinal axis of the object;
means for detection facing the means for providing a source of radiation;
means for control;
means for acquiring the signals transmitted by the means for detection;
means for image reconstruction;
means for display,
wherein the means for acquisition processes either:
a succession of data relating to a region of the object which is acquired by the means for detection during rotation of the means for support of the means for providing a source of radiation so that the means for reconstruction generates an image in a tomographic mode, or
a succession of data acquired by moving the means for horizontal support along a longitudinal direction of the means for horizontal without
rotating the means for support in order to form a silhouette radiographic
image of the object in a radiographic mode; and
means for selection between the tomographic mode or the radiographic mode and a radioscopic mode, in which the images displayed are successive projected radioscopic images of the selected region of the object in a plane parallel to the longitudinal axis of the object.

31. The apparatus according to claim 30 comprising means for controlling the acquisition of the projected radioscopic images of the selected region of the object.

32. The apparatus according to claim 31 wherein the means for control comprises a pedal.

33. The apparatus according to claim 30 comprising means for determining one or more radioscopic parameters as a function of the characteristics of a silhouette radiographic image of the object previously acquired.

34. The apparatus according to claim 31 comprising means for determining one or more radioscopic parameters as a function of the characteristics of a silhouette radiographic image of the object previously acquired.

35. The apparatus according to claim 32 comprising means for determining one or more radioscopic parameters as a function of the characteristics of a silhouette radiographic image of the object previously acquired.

36. The apparatus according to claim 30 comprising means for determining the radioscopic irradiation characteristics as a function of the characteristics of the successive radioscopic images of the selected region.

37. The apparatus according to claim 31 comprising means for determining the radioscopic irradiation characteristics as a function of the characteristics of the successive radioscopic images of the selected region.

38. The apparatus according to claim 32 comprising means for determining the radioscopic irradiation characteristics as a function of the characteristics of the successive radioscopic images of the selected region.

39. The apparatus according to claim 30 wherein the means for detection comprises a plurality of rows of radiation detectors extending along the longitudinal axis of the means for horizontal support.

40. The apparatus according to claim 31 wherein the means for detection comprises a plurality of rows of radiation detectors extending along the longitudinal axis of the means for horizontal support.

41. The apparatus according to claim 32 wherein the means for detection comprises a plurality of rows of radiation detectors extending along the longitudinal axis of the means for horizontal support.

42. The apparatus according to claim 33 wherein the means for detection comprises a plurality of rows of radiation detectors extending along the longitudinal axis of the means for horizontal support.

43. The apparatus according to claim 36 wherein the means for detection comprises a plurality of rows of radiation detectors extending along the longitudinal axis of the means for horizontal support.

44. The apparatus according to claim 37 wherein the means for detection comprises a plurality of rows of radiation detectors extending along the longitudinal axis of the means for horizontal support.

45. The apparatus according to claim 38 wherein the means for detection comprises a plurality of rows of radiation detectors extending along the longitudinal axis of the means for horizontal support.

46. The apparatus according to claim 30 wherein the means for providing a source of radiation comprises means for collimation for restricting the width of the field in the direction perpendicular to the longitudinal axis of the object.
